SEQUENCE LISTING (4 sequences)

SEQ ID NO: 1 - protein, 736 aa, Bacillus anthracis
MEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 2 - protein, 18 aa, Bacillus anthracis
IRKILSGYIVEIEDTEGL

SEQ ID NO: 3 - protein, 18 aa, Bacillus anthracis
RYDMLNISSLRQDGKTFI

SEQ ID NO: 4 - protein, 15 aa, Bacillus anthracis
FIDFKKYNDKLPLYI

(19) United States
(12) Patent Application Publication
(10) Pub. No.: US 2009/0317919 A1
(43) Pub. Date: Dec. 24, 2009

(54) Method for Assaying Antigens

(76) Inventors: Allan Watkinson, Guisborough (GB); Robert Wilson, Cumbernauld (GB); Steven Douglass, Tyne and Wear (GB); James Philip Myers, Bucks (GB)

Correspondence Address: CARELLA, BYRNE, BAIN, GILFILLAN, CECCHI,;STEWART & OLSTEIN, 5 BECKER FARM ROAD, ROSELAND, NJ, 07068, US

(21) Appl. No.: 12/226,101

(22) PCT Filed: Apr. 12, 2007
(86) PCT No.: PCT/GB2007/001353

§ 371 Date: Mar. 27, 2009

(30) Foreign Application Priority Data
Apr. 13, 2006 (GB) 0607462.9

(51) Int. Cl. G01N 33/566; G01N 33/543

(52) U.S. Cl. 436/501; 436/518

(57) ABSTRACT

A method for assaying an antigen adsorbed on a solid support is provided. The method comprises: a) contacting a suspension of the antigen adsorbed on the solid support with a solution or suspension of an optionally detectably-labelled primary antibody to the antigen to form a solid-supported antigen-antibody complex; b) where the primary antibody is detectably labelled, optionally measuring the detectable label thereby to determine the quantity of the antigen; or c) contacting the solid-supported antigen-antibody complex with a detectably-labelled probe for the solid-supported antigen-antibody complex; and d) measuring the detectable label on the probe, thereby to determine the quantity of antigen. The antigen is preferably a component of a sub-unit vaccine, and most preferably anthrax protective antigen.

The present invention concerns a method for assaying antigens, particularly components of sub-unit vaccines, and especially anthrax protective antigen, and more particularly to a method for measuring the viability or potency of said antigen.

Many vaccine drug products comprise an antigen adsorbed onto a solid inorganic support, especially a colloidal aluminium hydroxide, such as Alhydrogel. The adsorption onto the support means the antigen is not amenable to many conventional assay methods, such as immunoassays. Immunoassay methods have been proposed for assaying such drug products, but these rely upon either the antigen desorbing from the support, thereby risking damaging the antigen, or indirect methods of analysis, wherein a known amount of antibody is contacted with the antigen, the residual antibody determined, and hence the quantity of antigen determined by difference. For many antigens, and particularly in the case of anthrax protective antigen, the strength of the adsorption increases with storage, rendering the risk of damage on desorption even greater. It would therefore be desirable to identify alternative methods for assaying antigens adsorbed onto solid supports, especially a method which can serve as a reliable measure of viability or potency, and can be applied in vitro.

According to the present invention, there is provided a method for assaying an antigen adsorbed on a solid support, which comprises:
a) contacting a suspension of the antigen adsorbed on the solid support with a solution or suspension of an optionally detectably-labelled primary antibody to the antigen to form a solid-supported antigen-antibody complex;
b) where the primary antibody is detectably labelled, optionally measuring the detectable label thereby to determine the quantity of the antigen; or
c) contacting the solid-supported antigen-antibody complex with a detectably-labelled probe for the solid-supported antigen-antibody complex; and
d) measuring the detectable label on the probe, thereby to determine the quantity of antigen.

Antigens which can be assayed by the method of the present invention include components of subunit vaccines, usually recombinant protein-based subunit vaccines. Examples of such antigens include Hepatitis B protective antigens, Herpes Simplex Virus antigens, Influenza antigens, Congenital cytomegalovirus (CMV) antigens, Tuberculosis antigens, HIV antigens, Diphtheria antigens, Tetanus antigens, Pertussis antigens and Yersinia pestis protective antigens, such as antigens comprising one, two or more antigenic proteins. Most preferably, the antigen is an anthrax protective antigen.

Anthrax protective antigens which can be assayed by the method according to the present invention are well known in the art. Preferably, the anthrax protective antigen is recombinant protective antigen as described in WO02/04646, incorporated herein by reference, such as the antigen having the sequence given in FIG. 2 (Sequence ID No. 1).

Examples of solid supports onto which the antigen is adsorbed are preferably pharmacologically-acceptable adjuvants, especially colloidal adjuvants, such as calcium phosphate, aluminium phosphate, such as the colloidal aluminium phosphate available under the trade name “AdjuPhos”, and most preferably aluminium oxyhydroxide, most commonly known by the name “Alhydrogel”. Aluminium oxyhydroxide is also known as aluminium hydroxide or alum.

The antigen adsorbed on the solid support is assayed as a suspension in a liquid, most often an aqueous liquid. Most commonly the liquid is an aqueous buffer, such as a buffered saline solution, especially a phosphate-buffered saline solution or a Tris-buffered saline solution. When a buffer is employed, it preferably has a pH in the range of from 7 to 8, most preferably around 7.5. When the antigen adsorbed on the solid support is not already in the form of a suspension, such suspensions can readily be prepared using method known in the art.

Antibodies which can be employed as either primary antibodies or as probes in the method of the present invention are preferably monoclonal antibodies. In many embodiments, the primary antibody is selected to be an antibody for a region of anthrax protective antigen which is indicative of the activity of the antigen, and preferably is an antibody for Domain 4 of anthrax protective antigen. Especially preferred antibodies are antibodies which bind to the regions defined by amino acids 636 to 653 inclusive (IRKILSGYIVEIEDTEGL, Sequence ID No. 2), 660 to 677 inclusive (RYDMLNISSLRQDGKTFI, Sequence ID No. 3) or 676 to 690 inclusive (FIDFKKYNDKLPLYI, Sequence ID No. 4) of the antigen having the Sequence ID No. 1. Most preferred antibodies are antibodies for regions which comprise the aspartic acid residue at position 684 of the antigen having the Sequence ID No. 1. Methods of identifying suitable antibodies are known in the art. Examples of suitable primary antibodies include anti-protective antigen monoclonal antibody clone 2B18 (mouse) commercially available from United States Biological, anti-protective antigen monoclonal antibody clone RDI-TRK3BA16-C3 (clone C3; mouse) commercially available from RDI-Fitzgerald, anti-protective antigen monoclonal antibody clone RDI-TRK3BA16-105 mouse mAb commercially available from RDI-Fitzgerald, and anti-protective antigen monoclonal antibody clone RDI-TRK3BA16-106 mouse mAb commercially available from RDI-Fitzgerald.

The primary antibody may be employed as a suspension. Preferably, the primary antibody is employed as a solution, most often an aqueous solution, and most commonly as a solution in an aqueous buffer, such as a buffered saline solution, for example a phosphate-buffered saline solution or a Tris-buffered saline soon. In certain embodiments, the buffer employed may comprise a blocking buffer. When an aqueous buffer is employed, it preferably has a pH in the range of from 7 to 8, most preferably around 7.5. In many embodiments, the primary antibody is employed in a molar excess to the anticipated amount of antigen, such as up to a 2100 times excess, commonly up to a 500 times excess.

Probes for the primary antibody/antigen complex that may be employed include secondary antibodies for the primary antibody/antigen complex, and immunoglobulin binding proteins such as Protein A and Protein G. Preferably, secondary antibodies for the primary antibody/antigen complex are employed.

When a secondary antibody for the primary antibody/antigen complex is employed, the secondary antibody is preferably an antibody for the primary antibody portion of the complex. In this case, selection of the secondary antibody will depend upon the nature of the primary antibody, and in particular where the primary antibody was produced. Examples of combinations of secondary antibodies with primary antibodies are well known in the art. In many cases, when the primary antibody is monoclonal, it is often produced in mice, and when polyclonal, in goats or rabbits. Preferred secondary antibodies include IgG anti-mouse antibody and IgG anti-rabbit antibody.

The secondary antibody may be employed as a suspension. Preferably, the secondary antibody is employed as a solution, most often an aqueous solution, and most commonly as a solution in an aqueous buffer, such as a buffered saline solution, especially a phosphate-buffered saline solution or a Tris-buffered saline solution. In certain embodiments, the buffer employed may comprise a blocking buffer. When an aqueous buffer is employed, it preferably has a pH in the range of from 7 to 8, most preferably around 7.5. In many embodiments, the secondary antibody is employed in a molar excess to the anticipated amount of primary antibody/antigen complex, such as up to a 100 times excess, commonly up to a 20 times excess, for example in the range of from a 1.5 to 10 times excess.

Detectable labels, and the methods for detecting them are well known in the art, and include radiolabels, such as ¹²⁵I labels; fluorescent labels, such as fluorescein and FITC labels; biotinylation with avidin or streptavidin detection; and enzymic labels, such as horseradish peroxidase and alkaline phosphatase labels. The labels may be attached by methods known in the art.

The contact between the solid-supported antigen and the antibody/antibodies may conveniently be accomplished in a centrifuge tube. The use of a centrifuge tube allows convenient separation of the solid-supported antigen and solid-supported antibody/antigen complex from solutions or suspensions by centrifugation. In other embodiments, the contact between the solid-supported antigen and the antibody/antibodies may conveniently be accomplished in a well plate, commonly a 96-well plate. The dimensions of the well plate are conveniently selected so that the separation of the solid-supported antibody and solid-supported antibody/antigen complex from solutions or suspensions can be achieved by spinning down the plate such that the solid-supported antigen or antibody/antigen complex remains in the wells whilst the solutions or suspensions are removed, for example by decanting or by pipetting. Other methods of contact known in the art may also be employed if desired, for example by using filter plates (well plates in which the base of the well is a filter membrane) where separation can be achieved by filtration. In certain embodiments, good results have been achieved by removing fixed volumes of supernatants, especially by pipette, and most preferably by multi-channel pipette.

The method according to the present invention commonly employs washing solutions and blocking agents in accordance with conventional practice for immunoassays.

Blocking agents which can be employed are well known in the art. Preferred blocking agents include non-ionic surfactants, such as polyalkoxylated sorbitan alkanoates, for example Tween 20™, and non-specific proteins such as powdered milk protein (casein) and bovine serum albumin (BSA). A preferred blocking agent comprises a mixture of polyalkoxylated sorbitan alkanoate and casein and optionally BSA. In many embodiments, good results have been achieved using the blocking buffer solution commercially available from Invitrogen as part of its Western Breeze™ kit.

The present invention is illustrated without limitation by the following examples.

### EXAMPLE 1

Reagents

Sample—Drug product having an anthrax protective antigen concentration of 0.2 mg/mL was prepared as follows. A solution of 0.4 mg/mL anthrax protective antigen was prepared by diluting 0.5 mL of anthrax protective antigen solution in phosphate-buffered saline (PBS, pH 7.4) with a further 1.125 mL of PBS. 0.26 mL of alhydrogel suspension (Sigma A-8222) was mixed with 0.74 mL of 1.22% saline. To this was added 1 mL of the 0.4 mg/mL anthrax protective antigen solution followed by gentle mixing for 1 hour at room temperature to give the drug product.

Primary antibody—as detailed in Table 1 below.

Secondary antibody—Sigma F0257 anti-mouse IgG (whole molecule)-FITC antibody produced in goat (20 μg/mL i.e. 100 μL of conc. antibody in 10 mL of antibody diluent).

Blocking, antibody wash and antibody diluent solutions—from the Invitrogen Western Breeze kit and are prepared as per the Western Breeze kit instructions for PVDF membranes.

A Falcon 96-well tissue culture treated plate (transparent polystyrene) with a well volume of approximately 350 μL was used. 250 μL of blocking solution plus 5 μL of thoroughly mixed samples were loaded into wells in a randomised order. A normal single channel pipette of the Gilson type was used for the addition of samples to the plate. Additions of other solutions were done using a multi-channel pipette.

Mixing was done on a microplate shaker platform at a shaking speed of 500 rpm to avoid splashing. Spinning down was done using a Centurion centrifuge with a microplate rotor.

Supernatants were removed by removing the lid, turning the plate upside down to an angle of ca. 20° from the horizontal and gently shaking it up and down at a rate of approximately 1 cycle per second. The plate must not be vigorously shaken or flicked as the solid is only loosely bound to the base. Excessively vigorous shaking of the plate can dislodge some solid, reducing assay accuracy.

The sequence employed was as follows:

Immunoassay Procedure:

(1) Add 250 μL blocking solution to each well

(2) Add 5 μL of sample to each well

(3) Mix on the shaker platform at 500 rpm for 30 mins

(4) Spin down at 3 krpm for 5 mins

(5) Remove supernatant

(6) Add 200 μL of Primary Antibody Solution

(7) Mix on the shaker platform at 500 rpm for 1 hour

(8) Spin down at 3 krpm for 5 mins

(9) Remove supernatant

(10) Add 250 μL of Antibody Wash

(11) Spin down at 3 krpm for 5 mins

(12) Remove supernatant

(13) Add 250 μL of Antibody Wash

(14) Mix on the shaker platform at 500 rpm for 30 mins

(15) Spin down at 3 krpm for 5 mins

(16) Remove supernatant

(17) Add 250 μL of Water

(18) Spin down at 3 krpm for 5 mins

(19) Remove supernatant

(20) Add 200 μL of Secondary Antibody Solution

(21) Mix on the shaker platform at 50 rpm for 1 hour

(22) Spin down at 3 krpm for 5 mins

(23) Remove supernatant

(24) Add 250 μL of Antibody Wash

(25) Spin down at 3 krpm for 5 mins

(26) Remove supernatant

(27) Add 250 μL of Antibody Wash

(28) Mix on the shaker platform at 500 rpm for 30 mins

(29) Spin down at 3 krpm for 5 mins

(30) Remove supernatant

(31) Add 200 μL of 8M Urea to solubilise fluorescent label

(32) Mix on the shaker platform at 500 rpm for 30 mins

(33) Read the fluorescence of the wells using a plate reader.

The fluorescence was read using a Bio-TEK Synergy HT with an excitation wavelength of 485 nm and an emission wavelength of 528 nm.

To illustrate the utility of the method of the present invention as a measure of viability, the immunoassay procedure was used to assay three samples of anthrax protective antigen drug product. Two samples were products of storage stability trials and were known to have lost activity, and one sample was a sample from a storage stability trial known to have retained acceptable activity. As standards, a dilution series prepared from a recent batch of anthrax protective antigen drug product was prepared, together with a blank comprising the drug product diluent but no anthrax protective antigen. The three assay samples and the standard were all of the same formulation.

Three different primary antibodies were employed, as detailed in Table 2, each having been shown to bind to anthrax protective antigen domain 4.

Table 3 shows the samples which were assayed.

Six wells of each sample were assayed, two cells each of the three primary antibodies.

A Bio-TEK Synergy HT Plate reader was used for the fluorescence measurements.

TABLE 1
Primary Antibodies employed

| Abbreviation | Antibody | Concentration used |
|---|---|---|
| USBio | United States | 10 μg/mL i.e. 50 μL of |
| | Biological B0003-05J | conc. antibody in 10 mL |
| | Clone 2B18 mouse | of antibody diluent |
| | mAb | |
| RDI105 | RDI-TRK3BA16-105 | 10 μg/mL i.e. 12 μL of |
| | mouse mAb | conc. antibody in 10 mL |
| | | of antibody diluent |
| RDI106 | RDI-TRK3BA16-106 | 10 μg/mL i.e. 24.4 μL of |
| | mouse mAb | conc. antibody in 10 mL |
| | | of antibody diluent |

TABLE 2
Samples

| Sample Name | Description |
|---|---|
| 100% Std | Standard active drug product |
| 66% Std | Standard active drug product diluted to |
| | 66% activity with BPS formulated diluent |
| 33% Std | Standard active drug product diluted to |
| | 33% activity with BPS formulated diluent |
| Blank | BPS formulated diluent without any anthrax |
| | protective antigen |
| Sample 1 | Drug product from storage known to have |
| | retained acceptable activity |
| Sample 2 | Sample from a stability study know to have |
| | lost potency |
| Sample 3 | Sample from a second stability study know |
| | to have lost potency |

Results

The average fluorescence results for the three standard solutions and blank (shown in Table 3 below) were plotted to give standard curves for each antibody. The standard curves are shown in FIG. 1 below.

TABLE 3
Fluorescence Results for Standard solutions and Blank

| | Primary Antibody | | |
|---|---|---|---|
| | USBio | RDI 105 | RDI 106 |
| Sample | FI(RFU) | FI(RFU) | FI(RFU) |
| 100% Std (Batch 804151) | 13161 | 19087 | 12506 |
| 66% Std | 11333 | 13234 | 10293 |
| 33% Std | 8175 | 9459 | 7704 |
| Blank (i.e. diluent) | 6737 | 7520 | 5910 |

Using the mean fluorescence figures obtained for each of samples 1 to 3, the relative activity of each of the samples could be determined. The mean fluorescence figures, and percentage activity are given in Table 4 below.

TABLE 4
Fluorescence Results and Activity for Samples 1 to 3

| | Primary Antibody | | | | | |
|---|---|---|---|---|---|---|
| | USBio | | RDI 105 | | RDI 106 | |
| | | % | | % | | % |
| Sample | FI(RFU) | Activty | FI(RFU) | Activty | FI(RFU) | Activty |
| 1 | 12121 | 84% | 15669 | 79% | 11032 | 79% |
| 2 | 5700 | −12% | 7582 | 9% | 5756 | 0% |
| 3 | 4033 | −36% | 4011 | −22% | 3520 | −33% |

From the results, it can be seen that sample 1, which was known to have acceptable activity, gave a very good response compared with the active standard, whereas the samples known to have lost potency gave extremely poor results. This demonstrates that the method according to the present invention can be employed as an in vitro viability/potency assay for anthrax protective antigen.

### EXAMPLE 2

Samples of drug product from a 40° C. storage stability trials and a control stored at 2-8° C. were analysed by a variation on the method given in Example 1 above. The standard curve was generated by using a dilution series from standard material of known viability prepared by the same method which had been stored at 2-8° C. for 7 days.

All drug product and alhydrogel suspensions were handled by positive displacement pipettes.

The primary antibody employed was RDI-TRK3BA16-C3 (clone C3; mouse—commercially available from RDI-Fitzgerald) at 0.01 mg/ml concentration. The secondary antibody employed was Sigma F0257 anti-mouse IgG (whole molecule)-FITC antibody produced in goat at 6.246 mg/ml concentration (15 μl of supplied concentrate per 1 ml of diluent).

Blocking buffers and antibody washes employed were obtained from a Western breeze kit, supplied by Invitrogen.

The samples were diluted 1:2 with 0.26% alhydrogel in PBS, and this solution further diluted 1:24 with blocking buffer 125 μl of solution was added to wells of a Falcon 96-well tissue culture treated plate (transparent polystyrene) with a well volume of approximately 350 L.

The assay then followed the following procedure:

(1) Top up wells with 125 μl blocking buffer.

(2) Mix on the shaker platform at 500 rpm for 30 mins

(3) Spin down at 3 krpm for 5 mins

(4) Remove 150 μl supernatant using a multi-channel pipette

(5) Add 200 μL of Primary Antibody Solution

(6) Mix on the shaker platform at 500 rpm for 1 hour

(7) Spin down at 3 krpm for 5 mins

(8) Remove 200 μl supernatant using a multi-channel pipette

(9) Add 200 μL of Antibody Wash

(10) Spin down at 3 krpm for 5 mins

(11) Remove 200 μl supernatant using a multi-channel pipette

(12) Add 200 μL of Antibody Wash

(13) Mix on the shaker platform at 500 rpm for 5 mins

(14) Spin down at 3 krpm for 5 mins

(15) Remove 200 μl supernatant using a multi-channel pipette

(16) Add 200 μL of blocking buffer

(17) Spin down at 3 krpm for 5 mins

(18) Remove 200 μl supernatant using a multi-channel pipette

(19) Add 200 μL of blocking buffer

(20) Spin down at 3 krpm for 5 mins

(21) Remove 200 μl supernatant using a multi-channel pipette

(22) Add 200 μL of Secondary Antibody Solution

(23) Mix on the shaker platform at 500 rpm for 1 hour

(24) Spin down at 3 krpm for 5 mins

(25) Remove 200 μl supernatant using a multi-channel pipette

(26) Add 200 μL of Antibody Wash

(27) Spin down at 3 krpm for 5 mins

(28) Remove 200 μl supernatant using a multi-channel pipette

(29) Add 200 μL of Antibody Wash

(30) Mix on the shaker platform at 500 rpm for 5 mins

(31) Spin down at 3 krpm for 5 mins

(32) Remove 200 μl supernatant using a multi-channel pipette

(33) Add 200 μL of blocking buffer

(34) Spin down at 3 krpm for 5 mins

(35) Remove 200 μl supernatant using a multi-channel pipette

(36) Add 200 μL of blocking buffer

(37) Spin down at 3 krpm for 5 mins

(38) Remove 200 μl supernatant using a multi-channel pipette

The fluorescence was read using a Bio-TEK Synergy HT with an excitation wavelength of 485 nm and an emission wavelength of 528 nm.

The results of the assay showed that the control sample stored at 2-8° C. had an activity equivalent to 458.8 μl antigenic rPA/ml of drug product, whereas the sample stored at 40° C. had zero antigenic rPA remaining.

FIG. 2. Sequence of Anthrax Protective Antigen

(Sequence ID No. 1)

MEVKQENRLLNESESSSQGLLGYYFSDLNFQAPMVVTSSTTGDLSIPSSE
LENIPSENQYFQSAIWSGFIKVKKSDEYTFATSADNHVTMWVDDQEVINK
ASNSNKIRLEKGRLYQIKIQYQRENPTEKGLDFKLYWTDSQNKKEVISSD
NLQLPELKQKSSNSRKKRSTSAGPTVPDRDNDGIPDSLEVEGYTVDVKNK
RTFLSPWISNIHEKKGLTKYKSSPEKWSTASDPYSDFEKVTGRIDKNVSP
EARHPLVAAYPIVHVDMENIILSKNEDQSTQNTDSETRTISKNTSTSRTH
TSEVHGNAEVHASFFDIGGSVSAGFSNSNSSTVAIDHSLSLAGERTWAET
MGLNTADTARLNANIRYVNTGTAPIYNVLPTTSLVLGKNQTLATIKAKEN
QLSQILAPNNYYPSKNLAPIALNAQDDFSSTPITMNYNQFLELEKTKQLR
LDTDQVYGNIATYNFENGRVRVDTGSNWSEVLPQIQETTARIIFNGKDLN
LVERRIAAVNPSDPLETTKPDMTLKEALKIAFGFNEPNGNLQYQGKDITE
FDFNFDQQTSQNIKNQLAELNATNIYTVLDKIKLNAKMNILIRDKRFHYD
RNNIAVGADESVVKEAHREVINSSTEGLLLNIDKDIRKILSGYIVEIEDT
EGLKEVINDRYDMLNISSLRQDGKTFIDFKKYNDKLPLYISNPNYKVNVY
AVTKENTIINPSENGDTSTNGIKKILIFSKKGYEIG

1. A method for assaying an antigen adsorbed on a solid support, which comprises: a) contacting a suspension of the antigen adsorbed on the solid support with a solution or suspension of an optionally detectably-labeled primary antibody to the antigen to form a solid-supported antigen-antibody complex; b) where the primary antibody is detectably labeled, optionally measuring the detectable label thereby to determine the quantity of the antigen; or c) contacting the solid-supported antigen-antibody complex with a detectably-labeled probe for the solid-supported antigen-antibody complex; and d) measuring the detectable label on the probe, thereby to determine the quantity of antigen.

2. A method according to claim 1, wherein the antigen is a component of a sub-unit vaccine.

3. A method according to claim 2, wherein the antigen is anthrax protective antigen.

4. A method according to claim 3, wherein the primary antibody is an antibody for Domain 4 of anthrax protective antigen.

5. A method according to claim 2 wherein the solid support is selected from the group consisting of calcium phosphate, aluminum phosphate or aluminum oxyhydroxide.

6. A method according to any preceding claim, wherein the antigen adsorbed on the solid support is suspended in an aqueous buffer.

7. A method according to claim 6, wherein the buffer has a pH in the range of from 7 to 8.

8. A method according to claim 2, wherein the primary antibody is employed as a solution in an aqueous buffer.

9. A method according to claim 8, wherein the buffer has a pH in the range of from 7 to 8.

10. A method according to claim 2, wherein a detectably-labeled probe for the solid-supported antigen-antibody complex is employed.

11. A method according to claim 10, wherein the a detectably-labeled probe for the solid-supported antigen-antibody complex comprises a secondary antibody for the primary antibody/antigen complex.

12. A method according to claim 10, wherein the probe is employed as a solution in an aqueous buffer.

13. A method according to claim 12, wherein the buffer has a pH in the range of from 7 to 8.

14. A method according to claim 12, wherein the buffer has a pH about 7.5.

15. A method according to claim 12, wherein the buffer has a pH in the range of from 7 to 8.

16. A method according to claim 12, wherein the buffer has a pH around 7.5.

17. A method according to claim 6, wherein the buffer has a pH around 7.5.

18. A method according to claim 8, wherein the buffer has a pH about 7.5.

19. A method according to claim 11, wherein the probe is employed as a solution in an aqueous buffer.
